(19) United States
(12) Patent Application Publication
(10) Pub. No.: US 2014/0276165 A1
(43) Pub. Date: Sep. 18, 2014

(54) SYSTEMS AND METHODS FOR IDENTIFYING PATIENT TALKING DURING MEASUREMENT OF A PHYSIOLOGICAL PARAMETER

(71) Applicant: COVIDIEN LP, Mansfield, MA (US)

(72) Inventors: Paul S. Addison, Edinburgh (GB); James Watson, Dunfermline (GB)

(73) Assignee: Covidien LP, Mansfield, MA (US)

(21) Appl. No.: 13/829,012

(22) Filed: Mar. 14, 2013

(51) Int. Cl. A61B 5/08; A61B 7/00; A61B 5/00

(52) U.S. Cl. CPC A61B 5/08; A61B 5/746; A61B 7/003
USPC 600/529

(57) ABSTRACT

A patient monitoring system may include a microphone that generates a sound signal based on sound emanated from a patient. A patient monitoring unit may process the sound signal to identify respiration information such as respiration rate and to determine whether the patient was talking. If the patient was talking, a confidence value may be calculated, which may be used to generate a respiration information value.

The present disclosure relates to physiological signal processing, and more particularly relates to systems and methods for identifying patient talking during measurement of a physiological parameter.

### SUMMARY

A method for processing a sound signal comprises receiving a sound signal from a sensor that senses sound from a patient, determining, with processing equipment, respiration information based on the sound signal, identifying, with the processing equipment, a speech portion of the sound signal, determining, with the processing equipment, a confidence value associated with the respiration information based on the speech portion, and generating, with the processing equipment, a respiration information value based at least on the confidence value and the respiration information.

A non-transitory computer-readable storage medium for processing a sound signal has computer program instructions recorded thereon for receiving a sound signal from a sensor that senses sound from a patient, determining respiration information based on the sound signal, identifying a speech portion of the sound signal, determining a confidence value associated with the respiration information based on the speech portion, and generating a respiration information value based at least on the confidence value and the respiration information.

A monitoring unit comprises processing equipment configured to receive a sound signal from a sensor that senses sound from a patient, determine respiration information based on the sound signal, identify a speech portion of the sound signal, determine a confidence value associated with the respiration information based on the speech portion, and generate a respiration information value based at least on the confidence value and the respiration information.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system in accordance with some embodiments of the present disclosure;

FIG. 2 is a block diagram of the illustrative patient monitoring system of FIG. 1 coupled to a patient in accordance with some embodiments of the present disclosure;

FIG. 3 shows illustrative steps for determining respiration information and identifying talking of a patient in accordance with some embodiments of the present disclosure;

FIG. 4 shows illustrative steps for identifying patient talking in accordance with some embodiments of the present disclosure; and

FIG. 5 shows illustrative steps for determining respiration information in accordance with some embodiments of the present disclosure.

### DETAILED DESCRIPTION OF THE FIGURES

In a medical setting a sound signal may be generated by a microphone or other sensor based on sounds emanated by a patient. The sound signal may include speech, audible sounds, and other sounds that may not be audible but may be detected by an appropriate device.

Respiration is one physiological function that may be monitored by a patient monitoring system based on a sound signal generated by a microphone based on sounds emanated by a patient. While the patient is at rest, aspects of the sound signal unrelated to respiration may be minimal, or may be easily identified. When a patient is active or talking, it may be more difficult to identify aspects of the sound signal that relate to respiration, and the normal pattern of respiration may be changed based on the airflow due to the patient's activity (e.g., due to talking). In accordance with some embodiments of the present disclosure, portions of the sound signal that are related to talking may be identified by the patient monitoring system. The presence of patient talking may be indicated by patient monitor, for example on a display. In some embodiments, the processing of the sound signal to determine respiration may be modified to compensate for the patient talking.

For purposes of brevity and clarity, the present disclosure is written in the context of generating a sound signal based on sounds emanated from a patient, determining respiration information such as respiration rate from the sound signal, and identifying patient talking from the sound signal. It will be understood that any suitable physiological signal (e.g., photoplethysmograph (PPG), blood pressure, patient air flow, any other suitable signal, or any combination thereof) may be used in place of or in addition to the sound signal in accordance with the teachings of the present disclosure. It will also be understood that any other suitable physiological parameter may be determined in place of or in addition to respiration rate, or that an identification of patient talking may be performed in accordance with the teachings of the present disclosure without also determining additional physiological parameters. For purposes of brevity and clarity, the present disclosure refers to patient talking. Patient talking may refer to any audible sounds of a patient in addition to understandable speech.

FIG. 1 is a perspective view of an am embodiment of a patient monitoring system 10. System 10 may include monitoring unit 12 and a microphone 20. Monitoring unit 12 may provide for any suitable functionality, including user interface, data communications, interface with physiological sensors such as microphone 20, any other suitable functionality, or any combination thereof. Although a particular configuration of monitoring unit 12 is described herein it will be understood that the monitoring unit 12 may be implemented in any suitable manner.

In some embodiments, monitoring unit 12 may be implemented on a tablet-type computer unit, including a touch screen 14, speaker 16, power/wake button 18, and communication interface 24. It will be understood that any suitable device including suitable user interface, display, data inputs, and communication interfaces may be utilized in accordance with the present disclosure. In some embodiments, a personal computer, smart phone, or other standard computing device may implement the systems and methods described herein. In some embodiments, the systems and methods described herein may be implemented in a custom patient monitor, for example, to implement the specific functionality described herein or in combination with other patient monitoring functions.

As is described herein, monitoring unit 12 may analyze physiological information such as sound information received from microphone 20 to identify physiological information such as respiration rate and conditions such as patient talking. In some embodiments, a time series of respiration data related to inhalation and exhalation may be obtained and stored at monitoring unit 12. Although the respiration information may be determined from the respiration data in any suitable manner, in some embodiments, respiration information may be determined directly (e.g., by identifying sounds related to exhalation and inhalation), indirectly (e.g., by identifying changes in audible sounds due to respiration), in any other suitable manner, or any combination thereof. As is described herein, monitoring unit 12 may identify patient talking based on speech recognition, changes in respiration patterns, or a trained classifier (e.g., a classifier that identifies patient talking based on training information such as sound patterns that conform to patient talking, patient speech and sound characteristics, or any combination thereof).

Although a microphone is described herein, it will be understood that any other suitable sensor or combination of sensors may be used in addition to microphone 20 in accordance with the embodiments described herein. For example, in some embodiments respiration information such as respiration rate may be determined by one or more additional sensors in combination with or in place of microphone 20. Exemplary sensors may produce capnography signals, plethysmograph signals, trans-thoracic impedance signals, flow signals, thermistor signals, displacement signals (e.g., from chest or abdomen bands), any other suitable signals, or any combination thereof.

In some embodiments, touch screen 14 may provide a touch screen interface for users of monitoring unit 12. Although touch screen 14 may be configured in any suitable manner, in some embodiments, touch screen 14 may include menu 22, respiration waveform 26, respiration rate portion 28, alarm window 30, and patient alarm portion 32. Although monitoring unit 12 may be configured to determine any suitable physiological parameters based on any suitable sensor or data inputs, in some embodiments monitoring unit 12 may calculate respiration rate based on information received from microphone 20. In some embodiments respiration rate may be calculated at microphone 20, at an intermediate processing unit (not depicted), or at a remote processing unit (e.g., a remote computer or server) accessed via a communication link established by communication interface 24.

Microphone 20 may be any suitable microphone or combination of microphones that generates an electrical signal based on sounds received from a patient. Although microphone 20 is depicted as being physically coupled to monitor, it will be understood that electrical signals from microphone 20 may be transmitted to monitoring unit 12 in any suitable manner. In some embodiments, signals from microphone 20 may be transmitted wirelessly to an audio receiver of monitoring unit 12 (not depicted), converted to digital data and transmitted using standard communications protocols to monitoring unit 12 (e.g., via communication interface 24) (not depicted), or transmitted in any other suitable manner.

Microphone 20 may be located at any suitable location relative to a patient. In some embodiments, microphone may be located in a manner such that it is capable of receiving sounds related to patient airflow in addition to audible speech and noises. In some embodiments microphone 20 may be configured to receive a range of sounds including human speech, audible non-speech sounds, sounds that are directly indicative of respiration (e.g., airflow from breathing), modulations of speech or other human sounds caused by respiration, any other suitable sounds, or any combination thereof.

In some embodiments, respiration waveform 26 may be a waveform that is indicative of a patient's inhalation and exhalation as determined by monitoring unit 12. Respiration waveform 26 may be scaled in any suitable manner (e.g., based on selections of menu 22) for display, and may display real time data, stored respiration waveforms or other stored respiration information (e.g., a time-trend of respiration rate measurements) stored within memory of monitoring unit 12, or any other suitable information relating to respiration. It will also be understood that information relating to any other suitable physiological parameters may be displayed as a waveform in place of or in addition to respiration waveform 26.

Although any suitable physiological parameters may be displayed in accordance with the present disclosure, in some embodiments, a patient's respiration rate may be displayed on respiration rate display portion 28. Although a physiological parameter such as respiration rate may be displayed in any suitable manner, in some embodiments a value for the respiration rate may be displayed in breaths per minute, and the respiration rate portion 28 may flash when the calculated respiration rate falls outside of one or more predetermined limits (not depicted) which may be set, for example, by accessing menu 22. Although predetermined limit violations may be determined in any suitable manner, in exemplary embodiments the respiration rate limit may include an upper and lower limit. In some embodiments an alarm may be set to sound only after the alarm limit is violated for a predetermined time period, based on the degree of the respiration rate violation and the duration of the respiration rate violation, based on the rate of change of respiration rate, based on any other suitable parameters, or any combination thereof.

In some embodiments, patient alarm portion 32 may provide alarms or other notifications (e.g., as text notifications as depicted in the alarm portion 32 in FIG. 1) as well as an indication of the severity or degree of the condition that caused the alarm or notification (e.g., as depicted by the shaded bars of alarm portion 32 in FIG. 1). Although any suitable alarms or notification may be indicated within alarm portion 32, in some embodiments a notification of patient talking may be provided as a text notification and a severity indicator indicative of the impact that the patient talking has on the ability of the patient monitor to accurately determine a physiological parameter such as respiration rate. In some embodiments, different types of alarms relating to patient talking may be indicated by different alphanumeric displays, alarm colors, icons, or any combination thereof.

In some embodiments, alarm window 30 may overlay respiration waveform 26 to provide an indication of when an alarm or notification occurs relative to respiration waveform 26. Although any suitable alarms or notification may be indicated by alarm window 30 in this manner in accordance with the present disclosure, in some embodiments alarm window 30 may appear when the respiration rate falls outside of predetermined limits or when a patient is talking. In some embodiments, alarm window 30 may be displayed with respiration waveform 26 for recently received data, as well as for any respiration waveform 26 for stored respiration waveform data or respiration trend data (e.g., stored respiration rate trend data). Although alarm window 30 may be displayed in any suitable manner, in some embodiments alarm window 30 may be a shaded area that overlays the portion of respiration waveform 26 or respiration trend data that is associated with the alarm or notification. In some embodiments, different alarm types (e.g., respiration rate alarms, respiration rate upper limit alarms, respiration rate lower limit alarms, patient talking, and indications of patient talking that compromises the quality of the respiration rate determination) may be indicated in different manners, such as by changing the color of alarm window 30. Although multiple alarm or notification types may be displayed simultaneously in any suitable manner, in some embodiments, any portion of alarm window 30 that is associated with multiple alarms or notifications may display both colors simultaneously, for example, as interspersed colored bars within alarm window 30 (not depicted).

In some embodiments, alarm window 30 may be indicative of patient talking. Although an alarm window 30 related to patient talking may be displayed in any suitable manner, in some embodiments alarm window 30 may overlay a portion of a signal that coincides with a determination that the patient is talking. The alarm window 30 may also indicate severity of a talking condition, for example, based on the color, size, or shading of alarm window 30. In some embodiments, an alarm window 30 related to patient talking may be displayed with the respiration waveform 26 representing current respiration information of the patient. In some embodiments, an alarm window 30 may be displayed with respiration trend data relating to respiration information over time. For example, a respiration trend may provide in table or graphical form a representation of respiration trend data (e.g., respiration rate values over time). In an exemplary graphical representation, an alarm window 30 relating to patient talking may be displayed overlaying portions of the respiration trend that correspond to patient talking. In an exemplary table representation, an alarm window 30 relating to patient talking may be displayed by shading table entries that correspond to patient talking.

In some embodiments, menu portion 22 may include menus that allow a user to input data, adjust settings, change views, or interact with monitoring unit 12 in any suitable manner. In some embodiments, menu portion 22 may be implemented on touch screen 14, although it will be understood that menu portion 22 may be implemented in any suitable manner based on available user input options (e.g., buttons, keyboard, mouse, track pad, voice recognition, any other suitable user input, or any combination thereof) and display type of monitoring unit 12. Although menu portion 22 may include any suitable menus or information, in some embodiments menu portion 22 may include selectable menus for “menu,” “settings,” and “patient,” an informational area that includes messages to users (e.g., alarm information, help menus, and status information), and information such as time and date. The selectable menus of menu portion 22 may allow a user to adjust any suitable parameters and perform any suitable tasks for monitoring unit 12. Although any suitable functionality may be implemented by menu portion 22, in exemplary embodiments a user may be able to modify patient information, adjust alarm limits, define parameters to be measured, view or download stored data, and communicate with other devices (e.g., via voice, video, e-mail, or text messaging). In some embodiments, the options available through menu portion 22 may be based at least in part on a user's login credentials.

Although speaker 16 may be utilized in any suitable manner, in some embodiments, speaker 16 may provide audible sounds from monitoring unit 12 to enable monitoring unit 12 to communicate with a patient or medical professional and enable a user to communicate with other communication devices or users at other communication devices such as other patient monitors, nurse stations, mobile telephones, or any other suitable communication device. In some embodiments, speaker 16 may provide audible tones or messages in response to alarms or notifications (e.g., a notification of a low quality respiration signal caused by patient talking) as determined by monitoring unit 12. In some embodiments, the pitch, sound level, and duration of an alarm or notification may be modified based on alarm or notification type, alarm or notification duration, alarm or notification severity, any other suitable parameter related to alarms and notifications, or any combination thereof. In some embodiments, speaker 16 may provide spoken messages to a user, such as synthesized speech or prerecorded messages associated with alarms, indications of patient talking, and user inputs.

In some embodiments, communication interface 24 may provide for communication with devices external to monitoring unit 12. Although any suitable communication technologies may be implemented by communication interface 24, in some embodiments, communication interface 24 may include wired technologies (e.g., Ethernet, USB, FireWire, SCSI, and fiber networks), wireless technologies (e.g., WiFi, 3G networks, 4G networks, infrared, and radio frequency links), any other suitable communication technologies, or any combination thereof. It will be understood that any suitable communications with any suitable external devices may be performed via communication interface 24, such as data downloads, exchange of patient information, audio communications, video conferencing, and communication with other patient monitors and nurse stations.

FIG. 2 is a block diagram of a patient monitoring system, such as patient monitoring system 10 of FIG. 1, which may be coupled to a patient 40 in accordance with an embodiment of the present disclosure. Although this disclosure will be described with respect to a microphone measuring a sound signal, it will be understood that any suitable physiological measurement device may measure any suitable parameters in accordance with the present disclosure. Certain illustrative components of microphone 20 and monitoring unit 12 are illustrated in FIG. 2.

Microphone 20 may include transducer 70 and transmitter 72. Microphone 20 may be connected to a power source, for example via a wired connection with monitoring unit 12, or with an internal power source such as a battery (e.g., for a wireless microphone (not depicted)). It will be understood that microphone 20 may be any suitable microphone type based on any suitable transducer 70 type, such as condenser microphones, dynamic microphones, electret microphones, piezoelectric microphones, fiber optic microphones, laser microphones, micro electrical mechanical system (MEMS) microphones, any other suitable microphone, or any combination thereof. In some embodiments, multiple microphones 20, multiple transducer 70 types, or any combination thereof, may be selected to better identify different sound profiles (e.g., speech or respiration). Each transducer 70 may generate an electrical signal based on sound received at microphone 20, and an associated transmitter 72 may transmit the electrical signal output by transducer 70 to monitoring unit 12.

In some embodiments, one or more of the components described below with respect to monitoring unit 12 (e.g., amplifier 52, filter 54, A/D converter 56, any other suitable component, or any combination thereof) may be located at microphone 20. In this manner, the sound information received at transducer 70 may be processed or partially processed prior to being transmitted by transmitter 72 to receiver 50 of monitoring unit 12. In some embodiments, microphone 20 may include a processor and memory (not depicted) to perform data processing and transmitting functions (including some or all of amplifier 52, filter 54, A/D converter 56, any other suitable component, or any combination thereof). Although any suitable processing may be implemented at microphone 20, in some embodiments sound signals converted to electrical signals by transducer 70 and processed (e.g., by amplifier 52, filter 54, A/D converter 56, any other suitable component, or any combination thereof) may be converted into digital data for transmission to monitoring unit 12. Although sound information may be converted into digital data in any suitable manner, in some embodiments audio codecs, speech codecs, any other suitable speech processing technique, or any combination thereof, may be used to convert electrical sound information (e.g., due to respiration or speech) into digital data.

Signals from microphone 20 may be transmitted by transmitter 72 to a receiver 50 of monitoring unit 12. Although receiver 50 may receive any suitable sound signal in any suitable form, in some embodiments the received signal may be an electrical signal produced by transducer 70 of microphone 20 or digital data representing a sound signal. In some embodiments, receiver 50 or a plurality of receivers 50 may receive a plurality of signals associated with different sound profiles (e.g., due to respiration and speech) for independent or combined processing in accordance with the present disclosure.

In the embodiment shown, monitoring unit 12 may include a general-purpose microprocessor 62 connected to an internal bus 60. Microprocessor 62 may be adapted to execute software, which may include an operating system and one or more applications, as part of performing the functions described herein. Also connected to bus 60 may be a read-only memory (ROM) 64, a random access memory (RAM) 66, user inputs 68, display 20, communication interface 24, and speaker 16.

RAM 66 and ROM 64 are illustrated by way of example, and not limitation. Any suitable computer-readable media may be used in the system for data storage. Computer-readable media are capable of storing information that can be interpreted by microprocessor 62. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In some embodiments, the received signal from receiver 50 may be processed by amplifier 52, filter 54, and analog-to-digital converter 56. The digital data may then be stored in QSM 58 (or buffer) for later downloading to RAM 66 as QSM 58 is filled. In some embodiments, there may be multiple separate parallel paths for multiple received signals including additional components such as amplifier 52, filter 54, and/or A/D converter 56. Any suitable combination of components may be referred to herein as “processing equipment.”

In some embodiments, microprocessor 62 may determine respiration information from aspects of the sound signal relating to respiration. Respiration information may include respiration rate, which may be determined using various algorithms and/or look-up tables based on values calculated from the received signals and/or data corresponding to the signal or data received by receiver 50. Microprocessor 62 may generate a time series (trend) of respiration rate data from determined respiration rate values.

In some embodiments, microprocessor 62 may identify patient talking. Although patient talking may be identified in any suitable manner, in exemplary embodiments patient talking may be identified by performing speech recognition, by identifying talking based on changes in respiration patterns, by a trained classifier, or any combination thereof.

Although any suitable classifier may be used in accordance with the present disclosure, exemplary classifiers may include neural networks (e.g., maximum partial likelihood (MPL) networks or radial basis networks), genetic algorithms, stochastic and probabilistic classifiers (e.g., Basian, HMM, or fuzzy classifiers), propositional or predicate logics (e.g., non-monotonic or modal logics), nearest neighbor classification methods (e.g., k^{th} nearest neighbor or LVQ methods), any other suitable classifiers, or any combination thereof. Although any suitable signal processing techniques may be employed by the classifiers, exemplary signal processing techniques may include principal component analysis (PCA), independent component analysis (ICA), linear discriminate analysis (LDA), fast Fourier transforms, continuous wavelet transforms, Hilbert transforms, Laplace transforms, any other suitable signal processing method, or any combination thereof.

A classifier may be trained based on any suitable input parameters such as speech, sounds, respiration patterns, or any combination thereof. Training data may be any suitable data such as example data from a particular patient or a group of patients. Any portion of the training for the classifier may be performed at any suitable device at any suitable time. In some embodiments, the classifier may be trained entirely external to monitoring unit 12 and the classifier parameters may be stored at monitoring unit 12. In some embodiments, some or all of the training of the classifier may be performed at monitoring unit 12. For example, in some embodiments, parameters of the classifier may be updated for each patient. In some embodiments, the classifier may be continuously or periodically updated based on data received by monitoring unit 12 or by a number of monitoring units 12 (e.g., at a central monitoring station).

Although received data from a patient being monitored may be analyzed by a trained classifier in any suitable manner, in some embodiments one or more metrics may be determined based on sound data, respiration information, speech, any other suitable physiological parameter, or any combination thereof. As is described herein, the metrics may then be input to the classifier to output a classification. Any suitable classifications may be provided in accordance with the present disclosure, including classifications related to patient talking. In some embodiments, one or more classifications related to patient talking may indicate the severity of the patient talking.

In some embodiments, user inputs 68 may be used to enter information, select one or more options, provide a response, input settings, perform any other suitable input function, or any combination thereof. User inputs 68 may be used to enter information about the patient, such as age, weight, height, diagnosis, medications, treatments, and so forth. In some embodiments, display 14 may display values, data, alarms, menus, user messages, any other suitable information, or any combination thereof.

Communication interface 24 may provide for communications with other devices utilizing any suitable transmission medium as described herein. Communication interface 24 may receive messages to be transmitted from microprocessor 62 via bus 60. Exemplary data to be communicated may include respiration rate data, trend data, alarm information, indications of patient talking, speech signals, video signals, any other suitable information, or any combination thereof. In some embodiments, calculated metrics may be transmitted to an external device for determining one or more classifications based on determined metrics.

FIG. 3 is a flow diagram showing illustrative steps for determining respiration information and identifying talking in accordance with some embodiments of the present disclosure. In some embodiments, the steps described in FIGS. 3-5 figures may be performed by system 10. However, it will be understood that some or all of the steps of FIGS. 3-5 may be performed by one or more other devices such as a remote or networked patient monitor or central monitoring station.

At step 302, one or more microphones may generate a sound signal based on sound emanated by a patient. Although any suitable sound signal may be received by any suitable microphone or microphones, in some embodiments microphone 20 may include two transducers 70, with a first transducer configured to receive (higher frequency) speech and voice sounds and a second transducer configured to receive (lower frequency) respiratory sounds. First and second sound signals may be transmitted by transmitters 72 of microphone 40 to receivers 50 of monitoring unit 12. Although the received signals may be processed in any suitable manner, in some embodiments each of the received signals may be processed by an amplifier 52 and filter 54 tuned to isolate and identify the desired sounds associated with each signal (e.g., speech and respiration) before processing by A/D converter 56 and QSM 58, and storage at RAM 66 for processing by processor 62.

At step 304, processor 62 may identify any portions of the sound signal that are associated with patient talking. Although patient talking may be identified based on any suitable signal or combination of signals, in some embodiments an identification of talking may be identified based on a sound signal received at microphone 20. In some embodiments, processing to identify patient talking may be performed in accordance with the steps depicted in FIG. 4.

Referring to FIG. 4, at step 402, processor 62 may determine whether to use speech from the sound signal to identify patient talking. Although the determination of whether to use speech may be performed in any suitable manner, in some embodiments the determination may be based on the settings for monitoring unit 12, a signal quality for a speech portion of the received sound signal, any other suitable parameter related to the speech portion of the sound signal, or any combination thereof. If the speech signal is to be used to identify patient talking, processing may continue to step 404. If the speech signal is not to be used to identify patient talking, processing may continue to step 408.

At step 404, processor 62 may recognize speech from the sound signal. In some embodiments, words and phrases may be identified based on training data from a patient, for example, based on the patient speaking a number of predetermined words or phrases to assist in identifying patterns in the patient's speech. In some embodiments, the language spoken by the patient may be identified based on the training routine, a menu selection, any other suitable method, or any combination thereof. Although speech recognition may be performed in ay suitable manner, in some embodiments speech may be recognized based on Hidden Markov Models, neural networks, any other suitable speech recognition method, or any combination thereof.

At step 406, processor 62 may generate a confidence value based on the identification of speech in step 404. Although a confidence value may be determined in any suitable manner, in some embodiments a confidence value may be based on the percentage of the sound signal that includes talking, characteristics of the detected speech (e.g., pitch, volume), based on any other suitable parameter relating to the detected speech, or any combination thereof. A confidence value may be associated with a portion of the sound signal associated with the speech, and in some embodiments may be a value representative of the degree to which the patient talking is likely to impact the quality of the determination of respiration information from the sound signal.

At step 408, processor 62 may determine whether to identify patient talking based on changes in respiration determined from the sound signal. Although the determination of whether to use changes in respiration may be performed in any suitable manner, in some embodiments the determination may be based on the settings for monitoring unit 12, a signal quality for a portion of the received sound signal (e.g., a relevant frequency range associated with sounds of interest), any other suitable parameter related to respiration to be identified, or any combination thereof. If respiration is to be used to identify patient talking, processing may continue to step 410. If respiration is not to be used to identify patient talking, processing may continue to step 414.

At step 410, processor 62 may identify patient talking from respiration data determined from the sound signal. As is described herein, respiration data may be determined from the sound signal. Characteristics of the determined respiration data may itself be used to determine whether the patient is talking. For example, the breathing patterns of a patient may vary in a less repeatable fashion when a patient is talking than when a patient is not talking. During talking the patient's inhalations may become shorter while the exhalations may become relatively longer. Rather than being repeatable, the period of each breath may vary from breath to breath.

In some embodiments the distribution of the periods of the breaths for a set of respiration data may be determined. The spread of the distribution may be compared to a threshold, and a distribution that exceeds the threshold may be identified as based on patient talking. In some embodiments, the degree to which the spread of the distribution exceeds the threshold may be indicative of the severity of the patient talking condition (i.e., the degree to which the patient talking is likely to impact the quality of the respiration rate determination). In some embodiments, the threshold may be dependent on the mean respiration rate.

In some embodiments the ratio of the inhalation periods to the exhalation periods for a set of respiration data may be determined. This ratio may be compared to a threshold, and a ratio that falls below the threshold (i.e., indicates relatively short inhalation periods compared to exhalation periods) may be identified as based on patient talking. In some embodiments, the degree to which the ratio falls below the threshold may be indicative of the severity of the patient talking condition (i.e., the degree to which the patient talking is likely to impact the quality of the respiration rate determination). In some embodiments, the threshold may be dependent on the mean respiration rate.

In some embodiments the irregularity of the respiration waveform amplitude, or an envelope indicative of the respiration waveform amplitude, may be quantified. The variation in waveform amplitude may be compared to a threshold variation, and a variation that exceeds the threshold (i.e., indicates a high degree of variance in the respiration waveform) may be identified as based on patient talking. In some embodiments, the degree to which the variation exceeds the threshold may be indicative of the severity of the patient talking condition (i.e., the degree to which the patient talking is likely to impact the quality of the respiration rate determination). In some embodiments, the threshold may be dependent on the mean respiration rate.

At step 412, processor 62 may generate a confidence value based on the analysis of the respiration data in step 410. Although a confidence value may be determined in any suitable manner, in some embodiments a confidence value may be based on the percentage of the sound signal that includes talking, the severity of the patient talking condition, based on any other suitable parameter, or any combination thereof. In some embodiments a plurality of calculations relating to the respiration data may be performed as described herein, a confidence value may be calculated for each calculation, and the confidence values may be combined to generate a composite confidence value. The resulting confidence value may be associated with a portion of the sound signal that includes talking, and in some embodiments may be a value representative of the degree to which the patient talking is likely to impact the quality of the determination of respiration information from the sound signal.

At step 414, processor 62 may determine whether to use a classifier to identify patient talking. Although the determination of whether to use a classifier may be performed in any suitable manner, in some embodiments the determination may be based on the settings for monitoring unit 12, a signal quality for a portion of the received sound signal (e.g., a relevant frequency range associated with sounds of interest), any other suitable parameter related to the received sound signal, or any combination thereof. If a classifier is to be used to identify patient talking, processing may continue to step 416. If a classifier is not to be used to identify patient talking, processing may continue to step 422.

At step 416, processor 62 may calculate metrics to be input to the classifier. As described herein, the classifier may be trained based on training data. Metrics may be measurements that conform to the training data, and may be based on speech recognition, respiration data, any other suitable metric relating to the sound signal, or any combination thereof. It will be understood that any suitable number of metrics may be calculated from the received sound signal (or any data or signal obtained from the received sound signal), and that any number of metrics or combinations thereof may be input to any number of classifiers to identify patient talking. Examples of metrics include the frequency (pitch) of the sound(s), changes in the frequency (or tone) which may be used to inflect sound or speech patterns to convey emotional meaning, the amplitude (volume) and the change in amplitude of the signal which may indicate pain or stress, the appearance and disappearance of certain frequencies, ratios of the amplitudes of certain frequencies, the timbre (rise, duration and decay) of the sound signal components, or any other suitable metrics. These metrics may be computed from information extracted from the signal using a number of techniques. In some embodiments, the amplitude and frequency components of the signal may be extracted from the signal itself, or from the transform of a signal, including a Fourier or wavelet transform. In some embodiments, the metrics may be compared to a threshold which when exceeded may indicate patient distress. In some embodiments, the metrics may be input into a classifier which may have previously been trained on historic data. The classifier may use these metrics to indicate patient distress as described herein.

At step 418, processor 62 may process the metrics with one or more classifiers. As described herein, any suitable classifier may be used in accordance with the present disclosure, such as neural networks (e.g., MPL networks or radial basis networks), genetic algorithms, stochastic and probabilistic classifiers (e.g., Basian, HMM, or fuzzy classifiers), propositional or predicate logics (e.g., non-monotonic or modal logics), nearest neighbor classification methods (e.g., k^{th} nearest neighbor or LVQ methods), any other suitable classifiers, or any combination thereof. Although any suitable signal processing techniques may be employed by the classifiers, exemplary signal processing techniques may include PCA, ICA, LDA, fast Fourier transforms, continuous wavelet transforms, Hilbert transforms, Laplace transforms, any other suitable signal processing method, or any combination thereof.

In some embodiments, each classifier may output one or more values indicative of patient talking, the severity of patient talking on the determination of respiration, any other suitable values, or any combination thereof. In some embodiments, one or more of the classifiers may output logical values (e.g., “1” or “0”) indicative of the presence of a talking, severity values indicative of the presence and severity of talking, any other suitable values, or any combination thereof.

At step 420, processor 62 may generate a confidence value based on the output of the classifier or classifiers in step 418. Although a confidence value may be determined in any suitable manner, in some embodiments a confidence value may be based on the percentage of the sound signal that includes talking, the severity of the patient talking condition, based on any other suitable parameter, or any combination thereof. In some embodiments, a confidence value may be calculated based on each of a plurality of classifier outputs and the confidence values may be combined to generate a composite confidence value. The resulting confidence value may be associated with a portion of the sound signal that includes talking, and in some embodiments may be a value representative of the degree to which the patient talking is likely to impact the quality of the determination of respiration information from the sound signal.

At step 422, if multiple determinations of talking were made using different methodologies, the determinations may be combined. Although the determinations may be combined in any suitable manner, in some embodiments any regions of the sound signal associated with any of the determinations of talking may be assigned as a talking portion of the sound signal. At step 424, if more than one confidence value was determined for a single talking portion of the sound signal, the confidence values may be combined. Although the confidence values may be weighted and combined in any suitable manner, in some embodiments each confidence value may be weighted equally.

Referring again to FIG. 3, at step 306, processor 62 of monitoring unit 12 may determine respiration information such as respiration rate based on a received sound signal. Although respiration rate may be determined in any suitable manner, in some embodiments respiration rate may be determined in accordance with the steps of FIG. 5.

FIG. 5 shows illustrative steps for determining respiration information such as respiration rate in accordance with some embodiments of the present disclosure. Although it will be understood that respiration information may be calculated from any suitable signal or combination of signals (e.g., blood pressure signal, photoplethysmograph signal, air flow signal, motion signal (e.g., from measurements of body motion due to respiration), microphone (sound) signal, any other suitable signal, or any combination thereof), in some embodiments respiration information may be calculated based on a sound signal from microphone 20. Although any suitable techniques may be performed to determine respiration information, in exemplary embodiments monitoring unit 12 may perform respiration pre-processing, calculate respiration information, perform respiration post-processing, and communicate respiration information.

At step 502, processor 62 may perform respiration pre-processing on the received sound signal to generate a respiration signal. Although any suitable pre-processing techniques may be involved in respiration pre-processing, in some embodiments respiration pre-processing may be focused on distinguishing between portions of the sound signal that are likely to reflect data that is related to respiration and portions of the sound signal that are likely to reflect other information unrelated to respiration information, such as measurement error signal noise. For example, microphone 20 may be moved, temporarily located at a location remote from the desired location, or may encounter some other form of interference that degrades the signal or otherwise interferes with the identification of respiration information from the sound signal. In some embodiments, pre-processing may identify portions of the sound signal that fall outside of an acceptable value range for frequency, signal intensity, respiration rate, any other suitable parameter, or any combination thereof. Any portion of the sound signal that is identified may be compensated for in any suitable manner, for example, by excluding the data associated with the portion of the signal from determination of respiration information, down-weighting the data, or supplementing the data with respiration information available from other sources (e.g., based on modulations to a sound signal associated with speech or based on respiration signals determined from other measurement sources). In some embodiments the received sound signal for determining respiration information may be the same received sound signal for determining speech information (e.g., in an embodiment with a single received sound signal), and pre-processing for respiration may include filtering, for example, to emphasize sounds that are associated with respiration.

At step 504, processor 62 may calculate respiration information based on the pre-processed respiration signal. Although any suitable respiration information may be determined, in some embodiments the respiration information may be respiration rate. Although respiration rate may be determined in any suitable manner, in some embodiments the pre-processed respiration signal may be analyzed over time to determine a rolling average of a respiration rate. In some embodiments, a time series of respiration data from the pre-processed respiration signal may be analyzed to identify periodic aspects of the respiration signal, for example based on a Fourier transform, wavelet transform, performing an autocorrelation of the pre-processed respiration signal and identifying a period associated with a peak of the autocorrelation sequence, any other suitable technique for identifying periodic respiration information, or any combination thereof.

At step 506, processor 62 may perform post-processing based on the determined respiration rate. Although any suitable post-processing may be performed, in some embodiments the currently determined respiration rate may be combined with one or more recently determined respiration rates to determine a rolling average. In some embodiments, the averaging may be weighted based on the confidence value for the recently determined respiration rate. Although a confidence value may be determined in any suitable manner (e.g., as described in FIG. 4), in some embodiments a confidence value may also be based on the percentage of the respiration signal that was determined to include respiration information in pre-processing step 502, signal strength, a comparison of the most recently determined respiration rate value to previous respiration rate values, any other suitable measurement or determination, or any combination thereof. The result of the post-processing may be a respiration rate value, for example, for display and storage at monitoring unit 12.

Referring again to FIG. 3, at step 308, monitor 12 may generate a response based on the determination of respiration rate and determination of patient talking. Although any suitable response may be generated, in some embodiments monitor 12 may identify an audible response, a visual response, a message to be communicated to another device, an adjustment of any functionality that is integrated within monitor 12, any other suitable parameter, or any combination thereof. In some embodiments, these responses may include an audible response from speaker 16, an audible message from speaker 16, an alarm or notification on display 14, a message on display 14, communication with external devices via communication unit 24, updating data associated with an alarm window 30 corresponding to patient talking, or adjustment to treatments or parameters that are integrated within monitor 12.

In some embodiments, speaker 16 may provide an alarm or notification based on the respiration rate or determination of patient talking. Although alarms or notifications may be provided in any suitable manner, in some embodiments the tone, duration, sound level, any other suitable parameter, or any combination thereof, may be selected based on the severity of a respiration rate alarm or notification of patient talking.

In some embodiments, speaker 16 may provide an audible message based on the respiration rate or determination of patient talking. Although audible messages may be provided in any suitable manner, in some embodiments an audible message may be selected from one or more predetermined messages, for example, to indicate a respiration rate alarm, the presence of patient talking, or the severity of patient talking.

In some embodiments, an alarm may be indicated on display 14 based on the respiration rate or determination of patient talking. Although an alarm may be displayed in any suitable manner, in an exemplary embodiment an alarm type (e.g., patient talking or respiration alarm limit violation) and severity may be displayed within alarm portion 32. In some embodiments, a message may be indicated on display 14 based on the on the respiration rate or determination of patient talking, for example, within menu portion 22.

In some embodiments, monitor 12 may communicate with external devices via communication unit 24 based on the respiration rate or determination of patient talking. Although monitor 12 may communicate with any suitable devices, in some embodiments, monitor 12 may communicate with nurse stations, central monitoring units, remote servers, pagers, mobile telephones, medical devices, any other suitable device, or any combination thereof. For example, in an embodiment, in response to an indication of severe patient talking, monitoring unit 12 may communicate a message to a central monitoring station, to a pager of an attending physician. It will also be understood that any other suitable functionality may be integrated with monitor 12, such that in some embodiments the integrated functionality of monitor 12 may directly perform any suitable functionality in response to an indication of patient talking or a respiration rate alarm violation.

In some embodiments, data associated with an alarm window 30 may be updated based on an indication of patient talking. Although data associated with an alarm window 30 that is related to patient talking may be updated in any suitable manner, in an exemplary embodiment the portions of the respiration waveform and any respiration information determined during while the patient talking may be associated with a patient talking condition and stored in memory. In some embodiments, the associated patient talking condition may also indicate the severity of the patient talking condition. The alarm window 30 may then be displayed (e.g., with a current respiration waveform 26 or with respiration trend data) as described herein.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A method for processing a sound signal, the method comprising:
receiving a sound signal from a sensor that senses sound from a patient;
determining, with processing equipment, respiration information based on the sound signal;
identifying, with the processing equipment, a speech portion of the sound signal;
determining, with the processing equipment, a confidence value associated with the respiration information based on the speech portion; and
generating, with the processing equipment, a respiration information value based at least on the confidence value and the respiration information.

2. The method of claim 1, wherein identifying the speech portion of the sound signal comprises:
extracting a respiration signal from the sound signal;
identifying a plurality of breaths from the respiration signal;
determining a distribution of periods associated with the plurality of breaths; and
identifying the speech portion based at least in part on when the distribution exceeds a distribution threshold.

3. The method of claim 1, wherein identifying the speech portion of the sound signal comprises:
extracting a respiration signal from the sound signal;
identifying a plurality of breaths from the respiration signal;
determining an inhalation period for each of the plurality of breaths to produce a plurality of inhalation periods;
determining an exhalation period for each of the plurality of breaths to produce a plurality of exhalation periods;
calculating a ratio of the inhalation period and exhalation period for each of the plurality of breaths to produce a plurality of ratios; and
identifying the speech portion based on the plurality of ratios.

4. The method of claim 1, wherein identifying the speech portion of the sound signal comprises:
determining a classification of the sound signal based on a classifier, wherein the classifier is trained based on signal characteristics that correspond to vocal sounds; and
identifying the speech portion based on the classification.

5. The method of claim 4, wherein the classifier comprises one or more of a neural network, a genetic algorithm, stochastic classifiers, probabilistic classifiers, propositional logics, predicate logics, and nearest neighbor classification methods.

6. The method of claim 1, further comprising:
generating a respiration waveform; and
associating an alarm window with the respiration waveform based on the speech portion of the sound signal.

7. The method of claim 1, further comprising:
updating respiration trend data based on the respiration information; and
updating an alarm window associated with the respiration trend data based on the speech portion of the sound signal.

8. A non-transitory computer-readable storage medium for processing a sound signal, the computer-readable medium having computer program instructions recorded thereon for:
receiving a sound signal from a sensor that senses sound from a patient;
determining respiration information based on the sound signal;
identifying a speech portion of the sound signal;
determining a confidence value associated with the respiration information based on the speech portion; and
generating a respiration information value based at least on the confidence value and the respiration information.

9. The computer-readable medium of claim 8, wherein identifying the speech portion of the sound signal comprises:
extracting a respiration signal from the sound signal;
identifying a plurality of breaths from the respiration signal;
determining a distribution of periods associated with the plurality of breaths; and
identifying the speech portion based at least in part on when the distribution exceeds a distribution threshold.

10. The computer-readable medium of claim 8, wherein identifying the speech portion of the sound signal comprises:
extracting a respiration signal from the sound signal;
identifying a plurality of breaths from the respiration signal;
determining an inhalation period for each of the plurality of breaths to produce a plurality of inhalation periods;
determining an exhalation period for each of the plurality of breaths to produce a plurality of exhalation periods;
calculating a ratio of the inhalation period and exhalation period for each of the plurality of breaths to produce a plurality of ratios; and
identifying the speech portion based on the plurality of ratios.

11. The computer-readable medium of claim 8, wherein identifying the speech portion of the sound signal comprises:
determining a classification of the sound signal based on a classifier, wherein the classifier is trained based on signal characteristics that correspond to vocal sounds; and
identifying the speech portion based on the classification.

12. The computer-readable medium of claim 8, wherein the computer-readable medium has computer program instructions recorded thereon for:
generating a respiration waveform; and
associating an alarm window with the respiration waveform based on the speech portion of the sound signal.

13. The computer-readable medium of claim 8, wherein the computer-readable medium has computer program instructions recorded thereon for:
updating respiration trend data based on the respiration information; and
updating an alarm window associated with the respiration trend data based on the speech portion of the sound signal.

14. A monitoring unit comprises processing equipment configured to:
receive a sound signal from a sensor that senses sound from a patient;
determine respiration information based on the sound signal;
identify a speech portion of the sound signal;
determine a confidence value associated with the respiration information based on the speech portion; and
generate a respiration information value based at least on the confidence value and the respiration information.

15. The monitoring unit of claim 14, wherein the monitoring unit is configured to:
extract a respiration signal from the sound signal;
identify a plurality of breaths from the respiration signal;
determine a distribution of periods associated with the plurality of breaths; and
identify the speech portion based at least in part on when the distribution exceeds a distribution threshold.

16. The monitoring unit of claim 14, wherein the monitoring unit is configured to:
extract a respiration signal from the sound signal;
identify a plurality of breaths from the respiration signal;
determine an inhalation period for each of the plurality of breaths to produce a plurality of inhalation periods;
determine an exhalation period for each of the plurality of breaths to produce a plurality of exhalation periods;
calculate a ratio of the inhalation period and exhalation period for each of the plurality of breaths to produce a plurality of ratios; and
identify the speech portion based on the plurality of ratios.

17. The monitoring unit of claim 14, wherein the monitoring unit is configured to:
determine a classification of the sound signal based on a classifier, wherein the classifier is trained based on signal characteristics that correspond to vocal sounds; and
identify the speech portion based on the classification.

18. The monitoring unit of claim 17, wherein the classifier comprises one or more of a neural network, a genetic algorithm, stochastic classifiers, probabilistic classifiers, propositional logics, predicate logics, and nearest neighbor classification methods.

19. The monitoring unit of claim 14, wherein the monitoring unit is configured to:
generate a respiration waveform; and
associate an alarm window with the respiration waveform based on the speech portion of the sound signal.

20. The monitoring unit of claim 14, wherein the monitoring unit is configured to:
update respiration trend data based on the respiration information; and
update an alarm window associated with the respiration trend data based on the speech portion of the sound signal.
